(19) United States
(12) Patent Application Publication
(10) Pub. No.: US 2025/0169760 A1
(43) Pub. Date: May 29, 2025

(54) ADJUSTABLE UPPER LIMB WEARABLE DEVICE

(71) Applicant: National Cheng Kung University, Tainan (TW)

(72) Inventors: Chien-Hsiang Chang, Tainan (TW); Yang-Cheng Lin, Tainan (TW); Pei-Yun Wu, Tainan (TW); Yen-Chieh Kuo, Tainan (TW); Po-Ting Wu, Tainan (TW); Wei-Chih Lien, Tainan (TW); Bo Liu, Xiamen City (CN)

(21) Appl. No.: 18/926,851

(22) Filed: Oct. 25, 2024

(30) Foreign Application Priority Data
Nov. 29, 2023 (TW) 112146344

(51) Int. Cl. A61B 5/00; A63B 24/00; A63B 71/06

(52) U.S. Cl. CPC A61B 5/6824; A61B 5/4576; A61B 5/6831; A63B 24/0062; A63B 71/0622; A61B 2505/09; A63B 2024/0068

(57) ABSTRACT

The present invention provides an adjustable upper limb wearable device including: a main body having a first surface and a second surface, and the first surface is connected to the second surface to form a wearable structure. A positioning hole located on a connection line between the first surface and the second surface. A first sensor fixing bracket configured on the connection line adjacent to the positioning hole, and a first sensor is detachably configured within the first sensor fixing bracket. The adjustable upper limb wearable device can not only be worn easily, but also allow users to quickly position the sensor fixing brackets by aligning the positioning hole with the user's acromion to ensure the sensors are correctly positioned.

### CROSS REFERENCE

This non-provisional application claims benefit of Taiwan Patent Application No. 112146344 filed on Nov. 29, 2023. The contents thereof are incorporated by reference herein.

### FIELD OF THE INVENTION

This invention relates to an upper limb rehabilitation field, and particularly to an adjustable upper limb wearable device.

### BACKGROUND OF THE INVENTION

In the face of issues such as labor shortages and extending work time resulting from aging populations and declining birth rates, maintaining good health of labors and establishing solid exercise and training habits to prevent occupational injuries caused by overuse of the body become significantly important.

However, current wearable upper limb detecting devices on the market are not only inconvenient to use and wear, but also difficult to ensure the sensors are positioned correctly after the devices being worn. It will lead to data inaccuracies during detection and affect the accuracy of judgments made by healthcare professionals. Therefore, there is a need to develop upper limb detecting device that is more convenient to wear and can quickly ensure the sensors are correctly positioned.

### SUMMARY OF THE INVENTION

In one embodiment of the present disclosure provides an adjustable upper limb wearable device comprising: a main body having a first surface and a second surface, wherein the first surface is connected to the second surface to form a wearable structure; a positioning structure located on a connection line between the first surface and the second surface; a first sensor fixing bracket configured on the connection line and being adjacent to the positioning structure; a first sensor detachably configured within the first sensor fixing bracket.

Preferably, a center of the positioning structure is located on the connection line, and a shortest distance from the center of the positioning structure to a first projection center of the first sensor, projecting onto the main body, is in a range from 3 cm and 8 cm.

Preferably, the adjustable upper limb wearable device further comprises a second sensor fixing bracket configured on the connection line, the positioning structure is located between the first sensor fixing bracket and the second sensor fixing bracket, and a second sensor is detachably configured within the second sensor fixing bracket.

Preferably, a shortest distance from a second projection center of the second sensor fixing bracket projecting to a of the main body to the positioning structure is in a range from 8 cm to 13 cm.

Preferably, the adjustable upper limb wearable device further comprises an edge portion and a third sensor fixing bracket, the edge portion connects end points of the first surface, the second surface and the connection line, the edge portion is located on the main body opposite to the first sensor fixing bracket, the third sensor fixing bracket is configured on the second surface and adjacent to the edge portion, and a third sensor is detachably configured within the third sensor fixing bracket.

Preferably, a shortest distance between a third projection center of the third sensor fixing bracket projecting onto the main body and the edge portion is in a range from 3.5 cm to 6.5 cm.

Preferably, the adjustable upper limb wearable device further comprises a fourth sensor fixing bracket configured on the first surface and adjacent to the edge portion, and a fourth sensor is detachably configured within the fourth sensor fixing bracket.

Preferably, the adjustable upper limb wearable device further comprises a fifth sensor fixing bracket configured on the first surface and adjacent to the connection line, a fifth sensor is detachably configured within the fifth sensor fixing bracket, and a distance from the center of the positioning structure to a fifth projection center of the fifth sensor fixing bracket projecting onto the main body is greater than a distance from the center of the positioning structure to the first projection center of the first fixing bracket.

Preferably, an angle formed between a connecting line of a fourth projection center of the fourth sensor fixing bracket projecting onto the main body and the center of the positioning structure, and a connecting line of the fourth projection center and a second projection center of the second sensor fixing bracket projecting onto the main body, is 60 degrees.

Preferably, an angle between a connecting line of the third sensor fixing bracket projecting onto the main body and a second projection center of the second sensor fixing bracket projecting onto the main body, and a connecting line of the third projection center and the center of the positioning structure, is 15 degrees.

In summary, the adjustable upper limb wearable device provided in this disclosure is not only convenient to wear, but also can allow users to quickly position the sensor fixing brackets of the device by aligning the positioning structure with the acromion when wearing.

### BRIEF DESCRIPTION OF THE DRAWINGS

FIG. 1 is a schematic diagram illustrating an adjustable upper limb wearable device in one embodiment of the present disclosure.

FIG. 2 is a schematic diagram illustrating an adjustable upper limb wearable device in one embodiment of the present disclosure.

FIG. 3 is a schematic diagram illustrating the sensors in one embodiment of the present disclosure.

FIG. 4 is a schematic diagram illustrating the sensors in another embodiment of the present disclosure.

FIG. 5 is a schematic diagram illustrating an adjustable upper limb wearable device in another embodiment of the present disclosure.

FIG. 6 is a schematic diagram illustrating an adjustable upper limb wearable device in another embodiment of the present disclosure.

FIG. 7 is a schematic diagram illustrating an upper limb detecting system in one embodiment of the present disclosure.

### DETAILED DESCRIPTION OF THE INVENTION

In order to make the aforementioned and/or other purposes, benefits, and features of the present disclosure clearer and more understandable, the following detailed description is provided, using preferred embodiments as examples.

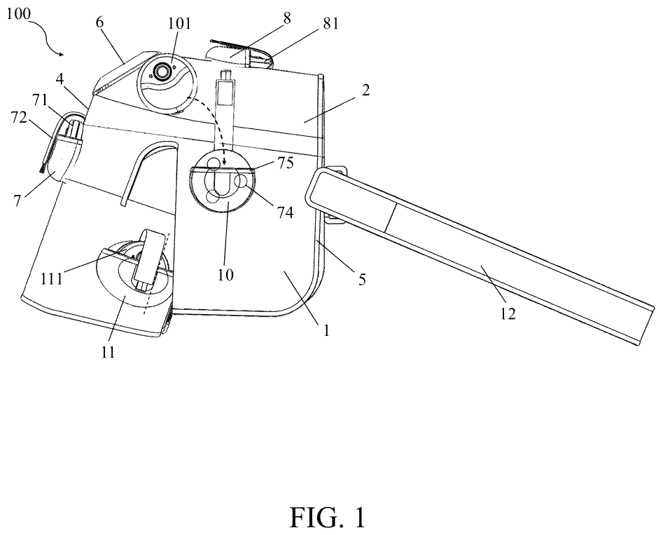

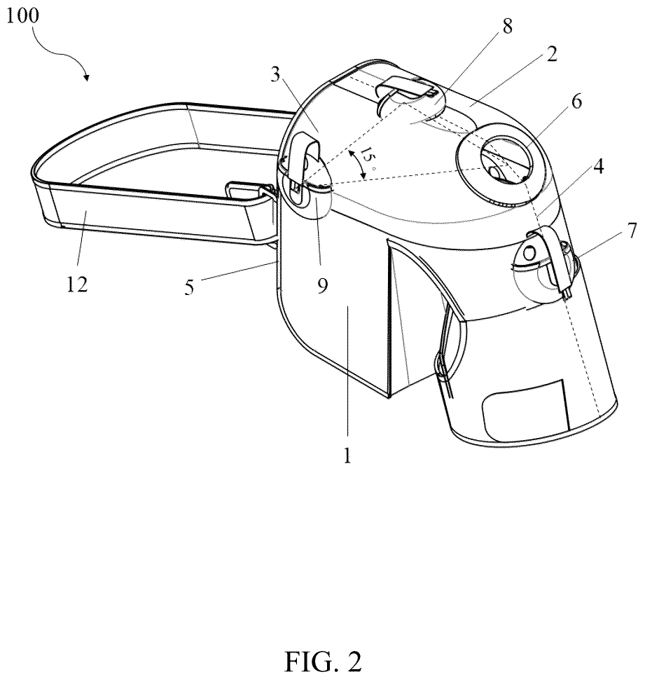

Please refer to FIGS. 1 and 2, FIG. 1 and FIG. 2 are schematic diagrams illustrating an adjustable upper limb wearable device in one embodiment of the present disclosure. The adjustable upper limb wearable device 100 includes a main body 1 having a first surface 2 and a second surface 3. The first surface 2 and the second surface 3 are connected to each other to form a wearable structure for the users to wear.

The main body 1 has a positioning structure 6. The positioning structure 6 is located on a connection line 4 between the first surface 2 and the second surface 3, and an opening of the positioning structure 6 penetrates through the main body 1. Preferably, the center of the positioning structure 6 is located on the connection line 4. When using, the positioning structure 6 corresponds to the user's acromion. After the user wears the adjustable upper limb wearable device 100, the sensors on the device can be positioned correctly by aligning the user's acromion with the positioning structure 6.

The main body 1 also includes an edge portion 5. The edge portion 5 is connected to the first surface 2 and the second surface 3, and the edge portion 5 is located at the end point of the connection line 4 between the first surface 2 and the second surface 3. When using, the edge portion 5 attaches to the user's back, shoulders and chest, and the edge portion 5 connected to the second surface 3 is parallel to the user's spine. Such that, it can facilitate the medical personnel to determine whether the user has correctly worn the adjustable upper limb wearable device 100. Furthermore, the edge portion 5 has an elastic fastener 12 configured to secure the adjustable upper limb wearable device 100 to fix on the user's body.

The adjustable upper limb wearable device 100 includes a first sensor fixing bracket 7. The first sensor fixing bracket 7 is configured on the connection line 4 between the first surface 2 and the second surface 3, and the first sensor fixing bracket 7 is adjacent to the positioning structure 6. The first sensor 71 can be detachably placed in the first sensor fixing bracket 7 and is configured to detect the physiological signals of the user's deltoid muscle. Preferably, the physiological signals detected by the first sensor 71 may be the surface electromyography signals, but the disclosure is not limited thereto, and users can choose the required sensors according to the trainings or measurements.

The first sensor fixing bracket 7 is equipped with a fixing strap 72 to fix the first sensor 71 in the first sensor fixing bracket 7 and to prevent the first sensor 71 from falling off. The fixing strap 72 can be fixed on the first sensor fixing bracket 7 by magnetic force, hook-and-loop fastener, or any other suitable means, and the present disclosure is not limited thereto.

In one embodiment, the projection center of the first sensor fixing bracket 7 projecting onto the main body 1 is located on the connection line 4, and the shortest distance from the center of the positioning structure 6 to the first sensor fixing bracket 7 is in a range from 3 cm to 8 cm.

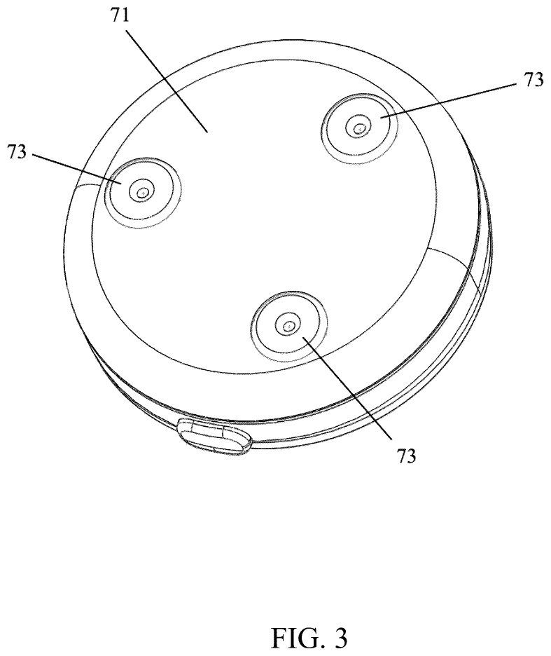

Please refer to FIG. 3, FIG. 3 is a schematic diagram illustrating the sensors in one embodiment of the present disclosure. The first sensor 71 includes three sensing elements 73 configured on the main body 1. The sensing element 73 is inserted through the through hole 74 and contacts with the user's skin. Among the three sensing elements 73 of the first sensor 71, the connecting line of two sensing elements is parallel to the connection line 4. These two sensing elements are used to collect the physiological signals of the user. The remaining sensing element is used to calibrate noise in the signals. After the user wears the adjustable upper limb wearable device 100, the connecting line of the two sensing elements, which are used to collect the physiological signals of the user, is parallel to the direction of the user's muscle fibers. In one example, within the first sensor 71, the connecting line of the two sensing elements used to collect the physiological signals of the user is parallel to the direction of the user's deltoid muscle fibers. In this disclosure, other sensors on the adjustable upper limb wearable device also have three sensing elements like the first sensor 71, and when the adjusted upper limb wearable device is worn by the user, the connecting line of the two sensing elements used to collect the physiological signals of the user is also parallel to the direction of the target muscle fibers.

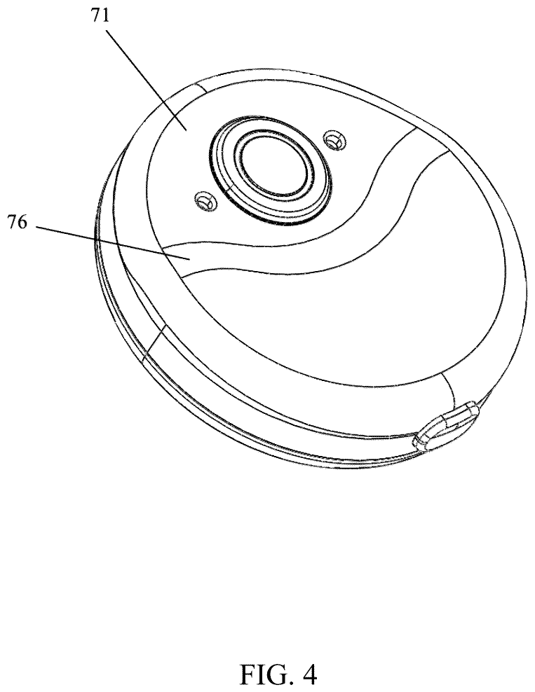

Please refer to FIG. 4, FIG. 4 is a schematic diagram illustrating the sensors in another embodiment of the present disclosure. The first sensor fixing bracket 7 further includes a positioning portion 75. The positioning portion 75 corresponds to the positioning element 76 of the first sensor 71. In one example, the positioning element 76 can be arc shaped. When the user places the first sensor 71 into the first sensor fixing bracket 7, the arc-shaped positioning element 76 of the first sensor 71 is aligned with the positioning portion 75 on the first sensor fixing bracket 7, such that the sensing elements on the first sensor 71 can be positioned and oriented correctly, and the sensing elements 73 can be installed correctly into the through hole 74 of the main body 1. This can prevent errors in collecting physiological signals caused by incorrectly positioning the sensing elements.

In one embodiment, the adjustable upper limb wearable device 100 further includes a second sensor fixing bracket 8 configured on the connection line 4. The positioning structure 6 is located between the first sensor fixing bracket 7 and the second sensor fixing bracket 8. The second sensor 81 can be detachably placed in the second sensor fixing bracket 8. When using, the second sensor 81 corresponds to the user's upper trapezius muscle and is configured to detect the physiological signals of the user's upper trapezius muscle.

Preferably, the projection center of the second sensor fixing bracket 8 projecting onto the main body 1 is located on the connection line 4, and the shortest distance from the projection center of the second sensor fixing bracket 8 and the positioning structure 6 is in a range from 8 cm to 13 cm.

In one embodiment, the adjustable upper limb wearable device 100 further includes a third sensor fixing bracket 9. The third sensor fixing bracket 9 is located on the second surface 3 and adjacent to the edge portion 5. The third sensor 91 can be detachably placed into the third sensor fixing bracket 9. When using, the third sensor 91 corresponds to the user's rhomboid muscle and is used to detect the physiological signals of the user's rhomboid muscle.

Preferably, the shortest distance from the projection center of the third sensor fixing bracket 9 projecting onto the main body 1 to the edge portion 5 is in a range from 3.5 cm and 6.5 cm.

In one embodiment, the adjustable upper limb wearable device 100 further includes a fourth sensor fixing bracket 10. The fourth sensor fixing bracket 10 is configured on the first surface 2 and adjacent to the edge portion 5. The fourth sensor 101 can be detachably placed into the fourth sensor fixing bracket 10. When using, the fourth sensor 101 corresponds to the user's pectoralis major muscle and is used to detect the physiological signals of the user's pectoralis major muscle.

Preferably, the shortest distance from the projection center of the fourth sensor fixing bracket 10, projecting onto the main body 1, to the connection line 4 of the first surface 2 and the second surface 3 is in a range from 10.5 cm and 15.5 cm.

In one embodiment, the adjustable upper limb wearable device 100 further includes a fifth sensor fixing bracket 11. The fifth sensor fixing bracket 11 is configured on the first surface 2 and is adjacent to the connection line 4. The distance from the center of the positioning structure 6 to the projection center of the fifth sensor fixing bracket 11 projecting onto the main body 1 is greater than the distance from the center point of the positioning structure 6 to the projection center of the first sensor fixing bracket 7 projecting onto the main body 1. The fifth sensor 111 can be detachably configured in the fifth sensor fixing bracket 11. When using, the fifth sensor 111 corresponds to the user's biceps brachii muscle and is used to detect the physiological signals of the user's biceps brachii muscle.

Preferably, the distance between the projection center of the fifth sensor fixing bracket 11 projecting onto the main body 1 and the center point of the positioning structure 6 is in a range from 17 cm and 22 cm.

In another embodiment, there are specific angles configured between the connecting lines formed by the sensors on the adjustable upper limb wearable device 100 and the center point of the positioning structure 6. For example, the angle between the line connecting the projection center of the third sensor fixing bracket 9 projecting onto the main body 1 and the projection center of the second sensor fixing bracket 9 projecting onto the main body 1, and the line connecting the projection center of the third sensor fixing bracket 9 projecting onto the main body 1 and the center point of the positioning structure 6, is 15 degrees.

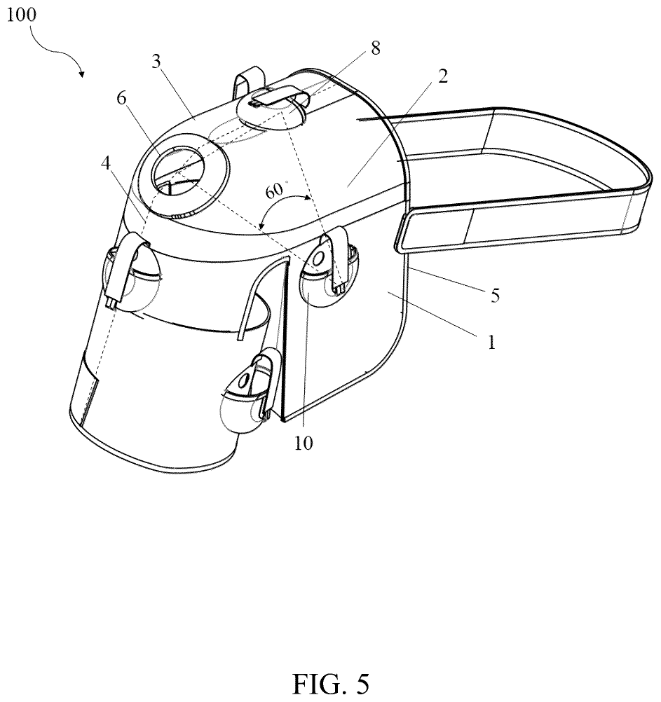

Alternatively, please refer to FIG. 5, the angle between the line connecting the projection center of the fourth sensor fixing bracket 10 projecting onto the main body 1 and the center point of the positioning structure 6, and the line connecting the projection center of the fourth sensor fixing bracket 10 projecting onto the main body 1 and the projection center of the second sensor fixing bracket 8 onto the main body, 1 is 60 degrees.

It should be noted that the second sensor fixing bracket 8, the third sensor fixing bracket 9, the fourth sensor fixing bracket 10, and the fifth sensor fixing bracket 11 are all similar to the first sensor fixing bracket 7, all having a fixing strap for securing the sensor. Therefore, further details regarding these components are not separately described herein.

In addition, the second sensor 81, the third sensor 91, the fourth sensor 101 and the fifth sensor 111 are all similar to the first sensor 71, including three sensing elements penetrating through the main body 1 and corresponds to the positioning element of the sensor fixing brackets. Further details regarding these components are not separately described herein.

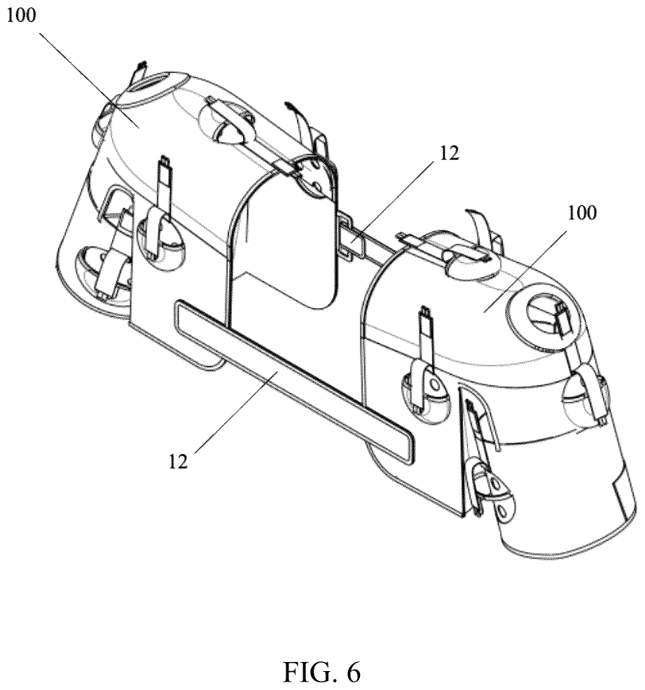

In another embodiment of the present invention, as shown in FIG. 6, two adjustable upper limb wearable devices 100 can form a set. When using, the two adjustable upper limb wearable devices 100 correspond to the user's shoulders respectively. In this embodiment, two elastic fasteners 12 connect the two adjustable upper limb wearable devices 100, allowing users to selectively adjust the length of the elastic fasteners 12 according to their body size to secure the two adjustable upper limb wearable devices 100 on their shoulders.

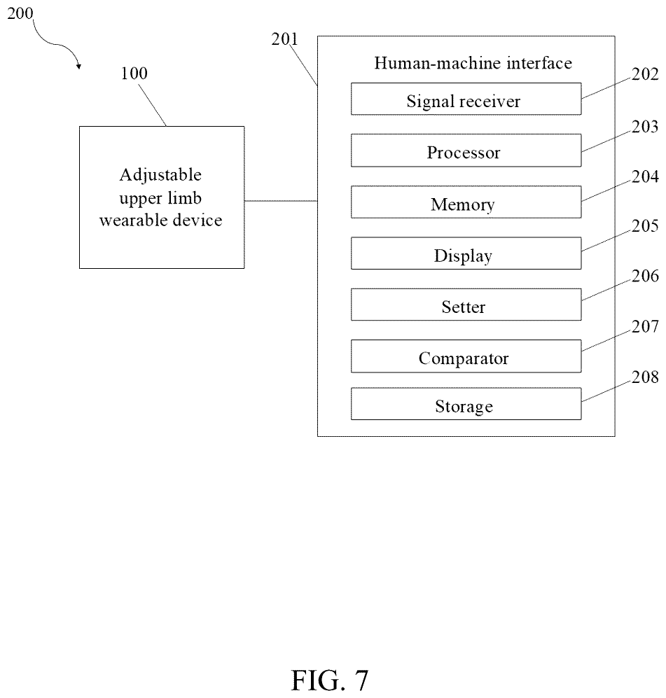

Please refer to FIG. 7, in another embodiment, the upper limb sensing system 200 includes at least one of the aforementioned adjustable upper limb wearable devices 100 and a human-machine interface 201. The adjustable upper limb wearable device 100 is wirelessly connected to the human-machine interface 201. For example, the adjustable upper limb wearable device 100 and the human-machine interface can be connected via Bluetooth.

The human-machine interface 201 includes a signal receiver 202, a processor 203, a memory 204, a display 205, a setter 206, a comparator 207 and a storage 208. Before the training and measurement, the medical personnel can set the training parameters via the setter 206. The processor 203 is configured to read the training content stored in the memory 204 and display it on the display 205 for the user to watch. The signal receiver 202 is configured to receive signals from the adjustable upper limb wearable device 100.

When the signal receiver 202 of the human-machine interface 201 receives the signals from the adjustable upper limb wearable device 100, the comparator 207 compares the received signal values with the values set by the medical personnel via the setter 206 to determine whether they are the same, and the comparator stores the comparison results in the storage 208. After the training process is completed, the medical personnel can read the comparison results stored in the storage 208 to assess the user's training performance and adjust the parameter values for the next training session accordingly.

In summary, the adjustable upper limb wearable device provided in this disclosure is not only convenient to wear, but also can allow users to quickly position the sensor fixing brackets of the device by aligning the positioning structure with the acromion when wearing. Such that, it can ensure that the sensors are in the correct position. Additionally, when placing the sensors into the sensor fixing brackets, the positioning elements can ensure that the sensors are installed in the correct orientation. This helps to avoid errors caused by installing the sensor at a wrong place or installing at an incorrect orientation. Therefore, the errors can be reduced, and the medical personnel may assess and analyze the data accurately.

However, the above description is merely a preferred embodiment of the present invention and should not be construed to limit the scope of the invention. Therefore, any simple equivalent changes and modifications made within the scope of the patent claims and the description of the invention are still within the scope of the patent coverage of the present invention.

What is claimed is:

1. An adjustable upper limb wearable device comprising:
a main body having a first surface and a second surface, wherein the first surface is connected to the second surface to form a wearable structure;
a positioning structure located on a connection line between the first surface and the second surface;
a first sensor fixing bracket configured on the connection line and being adjacent to the positioning structure;
a first sensor detachably configured within the first sensor fixing bracket.

2. The adjustable upper limb wearable device of claim 1, wherein the first sensor has three sensing elements, each sensing element is inserted through a through hole of the main body.

3. The adjustable upper limb wearable device of claim 2, wherein a connecting line of the two sensing elements is parallel to the connection line, and the two sensing elements are configured to collect the physiological signals.

4. The adjustable upper limb wearable device of claim 1, wherein the first sensor fixing bracket further comprises a positioning portion corresponding to a positioning element of the first sensor, when the first sensor is placed into the first sensor fixing bracket, the positioning element aligns with the positioning portion.

5. The adjustable upper limb wearable device of claim 1, wherein a center of the positioning structure is located on the connection line, and a shortest distance from the center of the positioning structure to a first projection center of the first sensor, projecting onto the main body, is in a range from 3 cm and 8 cm.

6. The adjustable upper limb wearable device of claim 1, wherein the adjustable upper limb wearable device further comprises a second sensor fixing bracket configured on the connection line, the positioning structure is located between the first sensor fixing bracket and the second sensor fixing bracket, and a second sensor is detachably configured within the second sensor fixing bracket.

7. The adjustable upper limb wearable device of claim 6, wherein a shortest distance from a second projection center of the second sensor fixing bracket projecting to a of the main body to the positioning structure is in a range from 8 cm to 13 cm.

8. The adjustable upper limb wearable device of claim 6, wherein the adjustable upper limb wearable device further comprises an edge portion and a third sensor fixing bracket, the edge portion connects end points of the first surface, the second surface and the connection line, the edge portion is located on the main body opposite to the first sensor fixing bracket, the third sensor fixing bracket is configured on the second surface and adjacent to the edge portion, and a third sensor is detachably configured within the third sensor fixing bracket.

9. The adjustable upper limb wearable device of claim 8, wherein a shortest distance between a third projection center of the third sensor fixing bracket projecting onto the main body and the edge portion is in a range from 3.5 cm to 6.5 cm.

10. The adjustable upper limb wearable device of claim 8, wherein the adjustable upper limb wearable device further comprises a fourth sensor fixing bracket configured on the first surface and adjacent to the edge portion, and a fourth sensor is detachably configured within the fourth sensor fixing bracket.

11. The adjustable upper limb wearable device of claim 10, wherein a shortest distance from a projection center of the fourth sensor fixing bracket projecting onto the main body to the connection line of the first surface and the second surface is in a range from 10.5 cm and 15.5 cm.

12. The adjustable upper limb wearable device of claim 10, wherein the adjustable upper limb wearable device further comprises a fifth sensor fixing bracket configured on the first surface and adjacent to the connection line, a fifth sensor is detachably configured within the fifth sensor fixing bracket, and a distance from the center of the positioning structure to a fifth projection center of the fifth sensor fixing bracket projecting onto the main body is greater than a distance from the center of the positioning structure to the first projection center of the first fixing bracket.

13. The adjustable upper limb wearable device of claim 12, wherein a distance from a projection center of the fifth sensor fixing bracket projecting onto the main body to the center point of the positioning structure is in a range from 17 cm and 22 cm.

14. The adjustable upper limb wearable device according to claim 10, wherein an angle formed between a connecting line of a fourth projection center of the fourth sensor fixing bracket projecting onto the main body and the center of the positioning structure, and a connecting line of the fourth projection center and a second projection center of the second sensor fixing bracket projecting onto the main body, is 60 degrees.

15. The adjustable upper limb wearable device of claim 14, wherein an angle between a connecting line of the third sensor fixing bracket projecting onto the main body and a second projection center of the second sensor fixing bracket projecting onto the main body, and a connecting line of the third projection center and the center of the positioning structure, is 15 degrees.

16. A upper limb sensing system, comprising:
at least one adjustable upper limb wearable device of claim 1;
a human-machine interface electrically connected to the at least one adjustable upper limb wearable device, wherein the human-machine interface comprises:
a signal receiver configured to receive signals from the at least on adjustable upper limb wearable device;
a memory;
a processor configured to read a training content stored in the memory;
a display configured to display the training content;
a setter;
a comparator; and
a storage;
wherein when the signal receiver receives the signals from the adjustable upper limb wearable device, the comparator compares the received signal values with pre-set values and stores the comparison results in the storage.
